(19) United States
(12) Patent Application Publication
(10) Pub. No.: US 2024/0173226 A1
(43) Pub. Date: May 30, 2024

(54) Oral Care Compositions

(71) Applicant: Colgate-Palmolive Company, New York, NY (US)

(72) Inventors: Robert D'AMBROGIO, Princeton, NJ (US); Jean DENIS, Union, NJ (US); Lauren EVANS, Highland Park, NJ (US)

(73) Assignee: Colgate-Palmolive Company, New York, NY (US)

(21) Appl. No.: 18/515,651

(22) Filed: Nov. 21, 2023

Related U.S. Application Data
(60) Provisional application No. 63/427,219, filed on Nov. 22, 2022.

(51) Int. Cl. A61K 8/27; A61K 8/21; A61K 8/24; A61K 8/25; A61K 8/44; A61K 8/73; A61Q 11/00

(52) U.S. Cl. CPC A61K 8/27; A61K 8/21; A61K 8/24; A61K 8/25; A61K 8/44; A61K 8/73; A61K 8/731; A61Q 11/00; A61K 2800/28; A61K 2800/48

(57) ABSTRACT

Described herein are oral care compositions comprising a surfactant system comprising an amino acid derived surfactant, and an amphoteric surfactant; a zinc salt; and a stannous ion source. Methods of making and using the same are also described.

### CROSS-REFERENCE TO RELATED PATENT APPLICATIONS

This application claims priority to U.S. Appl. No. 63/427,219, titled Oral Care Compositions and filed Nov. 22, 2022 the content of which is incorporated herein by reference in its entirety for all purposes.

### BACKGROUND

Dental erosion involves demineralization and damage to the tooth structure due to acid attack from nonbacterial sources. Erosion typically occurs initially in the enamel and, if unchecked, may proceed to the underlying dentin.

Dental erosion may be caused by or exacerbated by dental plaque. Dental plaque is a sticky biofilm or mass of bacteria that is commonly found between the teeth, along the gum line, and below the gum line margins. Dental plaque can give rise to dental caries and periodontal problems such as gingivitis and periodontitis. Dental caries, tooth decay, and/or tooth demineralization can be caused or exacerbated by acid produced from the bacterial degradation of fermentable sugar.

Stannous ion sources, such as stannous fluoride and stannous chloride, are known for use in clinical dentistry with a history of therapeutic benefits over forty years, and can have use in reducing certain bacterial growth in the oral cavity. However, until recently, the popularity of stannous ion sources has been limited by the instability in aqueous solutions. The instability of stannous salts in water is primarily due to the reactivity of the stannous ion (Sn²⁺). Stannous salts readily hydrolyze at a pH of above 4, resulting in precipitation from solution. It has traditionally been thought that this formation of insoluble stannous salts results in a loss of therapeutic properties. One common way to overcome the stability problems that can be associated with stannous ions is to limit the amount of water in the composition to very low levels, or to use a dual phase system.

Another ingredient that can be found in oral care compositions is sodium lauryl sulfate. Sodium lauryl sulfate can be useful as it may increase phase stability (e.g., reduces the likelihood of phase separation) of the oral care composition and generally does not affect the stability of the active ingredients in the oral care composition. In some cases, a surfactant substitution—adding another surfactant to replace sodium lauryl sulfate—has a negative impact on the taste or stability of the active ingredients. Moreover, microbiological stability of the formulation can be negatively impacted by the absence of sodium lauryl sulfate. There are also production benefits to having sodium lauryl sulfate in a given formulation. For example, by replacing sodium lauryl sulfate with other surfactants, the oral care composition may become overly aerated during production and it may be more difficult to clean the equipment after the production process.

There is an ongoing desire for improved oral care compositions.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to oral care compositions. In accordance with an aspect of the invention, provided is an oral care composition including a surfactant system comprising an amino acid derived surfactant, and an amphoteric surfactant; a zinc salt; and a stannous ion source.

In accordance with another aspect of the invention, provided is an oral care composition including from about 0.1 to about 4 wt. % of a surfactant system comprising an amino acid derived surfactant, and an amphoteric surfactant; a zinc salt; a stannous ion source; from about 5 to about 35 wt. % of a silica abrasive; from about 10 to about 50 wt. % of a polyol; and from about 0.5 to about 20 wt. % of a thickening agent, wherein all weight percentages are based on the total weight of the oral care composition.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the oral care composition. The symbol “°” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “−”, “=” and “=” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the oral care compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the oral care composition includes both components (or the composition includes more than two components that overlap), an overlapping compound does not represent more than one component. For example, stannous fluoride may be characterized as both a fluoride ion source and a stannous ion source. If a particular claim to an oral care composition includes both a fluoride ion source and a stannous ion source, stannous fluoride will serve only as either a fluoride ion source or a stannous ion source—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to oral care compositions and, e.g., oral care compositions providing antibacterial efficacy. Certain oral care compositions advantageously provide antibacterial efficacy and desirable metal update, e.g., on soft oral tissues. It was unexpectedly discovered that these benefits as well as others can be achieved with certain oral care compositions disclosed herein without the use of sodium lauryl sulfate.

In accordance with an aspect of the invention, provided is an oral care composition including a surfactant system comprising an amino acid derived surfactant, and an amphoteric surfactant; a zinc salt; and a stannous ion source. In accordance with another aspect of the invention, provided is an oral care composition including from about 0.1 to about 4 wt. % of a surfactant system comprising an amino acid derived surfactant, and an amphoteric surfactant; a zinc salt; a stannous ion source; from about 5 to about 35 wt. % of a silica abrasive; from about 10 to about 50 wt. % of a polyol; and from about 0.5 to about 20 wt. % of a thickening agent, wherein all weight percentages are based on the total weight of the oral care composition.

The oral care compositions may be formulated to have improved phase stability, while utilizing minimal or no sodium lauryl sulfate. For example, the amount of sodium lauryl sulfate present in the oral care composition may be about 5 wt. % or less, about 4 wt. % or less, about 3 wt. % or less, about 2 wt. % or less, about 1 wt. % or less, about 0.5 wt. % or less, about 0.1 wt. % or less, or about 0.05 wt. % or less. In some embodiments, the oral care composition is substantially free of or free of sodium lauryl sulfate. Additionally or alternatively, the oral care composition may have sodium laureth sulfate in an amount of about 5 wt. % or less, about 4 wt. % or less, about 3 wt. % or less, about 2 wt. % or less, about 1 wt. % or less, about 0.5 wt. % or less, about 0.1 wt. % or less, or about 0.05 wt. % or less. In some embodiments, the oral care composition is substantially free of or free of sodium laureth sulfate. In at least one embodiment, the oral care composition contains 0 wt. % or about 0 wt. % of sodium lauryl sulfate and/or sodium laureth sulfate.

The oral care compositions may be in the form of dentifrice, toothpaste, mouthwash, and/or the like. Suitable components, such as those listed below, may be included or excluded from the formulations for the oral care compositions depending on the specific combination of other ingredients and the form of the oral care compositions.

The oral care compositions typically include a surfactant system comprising an amino acid derived surfactant and an amphoteric surfactant. The surfactant system may be present in the oral care composition in an amount from about 0.5 to about 9 wt. %, based on the total weight of the oral care composition. In some embodiments, the oral care composition includes a surfactant system in an amount from about 0.5 to about 7 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 9 wt. %, about 1 to about 7 wt. %, about 1 to about 6 wt. %, about 1 to about 5 wt. %, about 1 to about 4 wt. %, about 1 to about 3 wt. %, about 1 to about 2 wt. %; from about 1.4 to about 9 wt. %, about 1.4 to about 7 wt. %, about 1.4 to about 6 wt. %, about 1.4 to about 5 wt. %, about 1.4 to about 4 wt. %, about 1.4 to about 3 wt. %, about 1.4 to about 2 wt. %; from about 1.8 to about 9 wt. %, about 1.8 to about 7 wt. %, about 1.8 to about 6 wt. %, about 1.8 to about 5 wt. %, about 1.8 to about 4 wt. %, about 1.8 to about 3 wt. %, about 1.8 to about 2 wt. %; from about 2.2 to about 9 wt. %, about 2.2 to about 7 wt. %, about 2.2 to about 6 wt. %, about 2.2 to about 5 wt. %, about 2.2 to about 4 wt. %, about 2.2 to about 3 wt. %; from about 3 to about 9 wt. %, about 3 to about 7 wt. %, about 3 to about 6 wt. %, about 3 to about 5 wt. %; from about 4 to about 9 wt. %, about 4 to about 7 wt. %, about 4 to about 6 wt. %; from about 5 to about 9 wt. %, about 5 to about 7 wt. %; from about 6 to about 9 wt. %, about 6 to about 8 wt. %, or any range or subrange thereof, based on the total weight of the oral care composition. The surfactants of the surfactant system, such as those disclosed below, may be present in the oral care composition in a salt form, a dissociated form, or a combination thereof.

The surfactant systems typically comprise two or more surfactants. For example, the surfactant system may include two, three, four, five, six, seven, eight, or nine or more surfactants. In some embodiments, the surfactant system comprises from two to eight, from two to seven, from two to six, from two to five, from two to four, from three to seven, from three to six, from three to five, from four to eight, four to seven, or four to six surfactants. Although the surfactant system typically comprises an amino acid derived surfactant and an amphoteric surfactant, the surfactant system may also comprise one or more anionic surfactant, one or more cationic surfactant, one or more nonionic surfactants, one or more amphoteric surfactants, one or more zwitterionic surfactants, or combinations of two or more thereof. For example, the oral care composition may have a surfactant system comprising an amino acid derived surfactant, an amphoteric surfactant, and a nonionic surfactant system. In some embodiments, the surfactant system consists of one or more amino acid derived surfactant, one or more amphoteric surfactant, and optionally one or more nonionic surfactant system. In further embodiments, the surfactant system consists of one or more amino acid derived surfactant, one or more amphoteric surfactant, and one or more nonionic surfactant system. In additional embodiments, the surfactant system consists of one or more amino acid derived surfactant and one or more amphoteric surfactant. Yet, in further embodiments, the surfactant system consists of one or more amino acid derived surfactant, one or more nonionic surfactant system, and optionally one or more amphoteric surfactant. In yet additional embodiments, the surfactant system consists of one or more amino acid derived surfactant and one or more nonionic surfactant system.

The surfactant system typically comprises one or more amino acid derived surfactant(s). The amount of amino acid derived surfactant(s) present in the oral care composition may vary, but can be from about 0.1 to about 4 wt. %, based on the total weight of the oral care composition. In some embodiments, the amount of amino acid derived surfactants in the oral care composition is from about 0.1 to about 3 wt. %, about 0.1 to about 2 wt. %, about 0.1 to about 1 wt. %; from about 0.3 to about 4 wt. %, about 0.3 to about 3 wt. %, about 0.3 to about 2 wt. %, about 0.3 to about 1 wt. %; from about 0.5 to about 4 wt. %, about 0.5 to about 3 wt. %, about 0.5 to about 2 wt. %, about 0.5 to about 1 wt. %; from about 0.8 to about 4 wt. %, about 0.8 to about 3 wt. %, about 0.8 to about 2 wt. %; from about 1.1 to about 4 wt. %, about 1.1 to about 3 wt. %, about 1.1 to about 2 wt. %; from about 1.5 to about 4 wt. %, about 1.5 to about 3 wt. %, about 1.5 to about 2 wt. %; from about 2 to about 4 wt. %, about 2 to about 3 wt. %; from about 2.5 to about 4 wt. %, about 2.5 to about 3 wt. %, or any range or subrange thereof, based on the total weight of the oral care composition.

The amino acid derived surfactant may be anionic, non-ionic, amphoteric, or cationic. In some embodiments, the amino acid derived surfactant is an anionic surfactant. The amino acid derived surfactant may be based on alanine, arginine, aspartic acid, glutamic acid, glycine, isoleucine, leucine, lysine, phenylalanine, serine, tyrosine, valine, sarcosine, threonine, and taurine. The amino acid derived surfactants disclosed herein may be present in the oral care composition in a salt form. The cation associated with acyl amino acid(s) can be sodium or potassium. Alternatively, the cation can be an organic salt such as triethanolamine (TEA) or a metal salt.

The amino acid derived surfactant may be an acyl amino acid derived surfactant having an aliphatic carbon chain of 3 to 21 carbons. For instance, the amino acid derived surfactant may include an alkyl group comprised of a carbon chain of 3 to 21 carbons, 3 to 19 carbons, 3 to 17 carbons, 3 to 15 carbons, 3 to 13 carbons, 3 to 11 carbons, 3 to 9 carbons, 3 to 7 carbons; 4 to 21 carbons, 4 to 19 carbons, 4 to 17 carbons, 4 to 15 carbons, 4 to 13 carbons, 4 to 11 carbons, 4 to 9 carbons, 4 to 7 carbons; 6 to 21 carbons, 6 to 19 carbons, 6 to 17 carbons, 6 to 15 carbons, 6 to 13 carbons, 6 to 11 carbons, 6 to 9 carbons; 9 to 21 carbons, 9 to 19 carbons, 9 to 17 carbons, 9 to 15 carbons, 9 to 13 carbons, 9 to 11 carbons; 11 to 21 carbons, 11 to 19 carbons, 11 to 17 carbons, 11 to 15 carbons, 11 to 13 carbons; 13 to 21 carbons, 13 to 19 carbons, 13 to 17 carbons, or any range or subrange thereof.

Preferably, the amino acid derived surfactant comprises a glutamic acid derived surfactant. The glutamic acid derived surfactant may be selected from an acyl glutamic acid derived surfactants having an alkyl group comprised of a carbon chain having 3 to 21 carbons or any of the ranges for the carbon chain described above with respect to the amino acid derived surfactant. For instance, the carbon chain of the acyl glutamic acid derived surfactant may be 4 to 19 carbons, optionally 6 to 17 carbons, or optionally 9 to 13 carbons. In some preferred embodiments, the amino acid derived surfactant comprises sodium cocoyl glutamate.

The surfactant system typically comprises one or more amphoteric surfactant(s). The amount of amphoteric surfactant(s) present in the oral care composition may vary, but can be from about 0.3 to about 8 wt. %, based on the total weight of the oral care composition. For example, the amount of amphoteric surfactant(s) may be from about 0.3 to about 7 wt. %, about 0.3 to about 6 wt. %, about 0.3 to about 5 wt. %, about 0.3 to about 4 wt. %, about 0.3 to about 3 wt. %, about 0.3 to about 2 wt. %; from about 0.6 to about 8 wt. %, about 0.6 to about 6 wt. %, about 0.6 to about 5 wt. %, about 0.6 to about 4 wt. %, about 0.6 to about 3 wt. %, about 0.6 to about 2 wt. %; from about 0.9 to about 8 wt. %, about 0.9 to about 6 wt. %, about 0.9 to about 5 wt. %, about 0.9 to about 4 wt. %, about 0.9 to about 3 wt. %, about 0.9 to about 2 wt. %; from about 1.2 to about 8 wt. %, about 1.2 to about 6 wt. %, about 1.2 to about 5 wt. %, about 1.2 to about 4 wt. %, about 1.2 to about 3 wt. %, about 1.2 to about 2 wt. %; from about 1.5 to about 8 wt. %, about 1.5 to about 6 wt. %, about 1.5 to about 5 wt. %, about 1.5 to about 4 wt. %, about 1.5 to about 3 wt. %; from about 2 to about 8 wt. %, about 2 to about 6 wt. %, about 2 to about 5 wt. %, about 2 to about 4 wt. %, about 2 to about 3 wt. %; from about 3 to about 8 wt. %, about 3 to about 6 wt. %, about 3 to about 5 wt. %; from about 4 to about 8 wt. %, about 4 to about 6 wt. %, about 4 to about 5 wt. %; from about 6 to about 8 wt. %, about 7 to about 8 wt. %, or any range or subrange thereof, based on the total weight of the oral care composition.

Amphoteric surfactants are typically characterized by a combination of high surfactant activity, lather forming and mildness. The amphoteric surfactant may comprise a substituent containing 8 to 18 carbon atoms and a substituent containing one or more carboxylate, sulfonate, sulfate, phosphate, or phosphonate. For instance, the amphoteric surfactant may have an alkyl group comprising from 8 to 20 carbon atoms, 8 to 16 carbon atoms, 10 to 16 carbon atoms, or 10 to 13 carbon atoms. The amphoteric surfactant(s) may include, but are not limited to, derivatives of aliphatic secondary and tertiary amines in which the aliphatic radical can be straight chain or branched. In some cases, one of the aliphatic substituents of the amphoteric surfactant contains about 8 to about 18 carbon atoms and one of the aliphatic substituents contains an anionic water solubilizing group, e.g., carboxy, sulfonate, sulfate, phosphate, or phosphonate. The amphoteric surfactants disclosed herein may be present in the oral care composition in a salt form.

The amphoteric surfactants may include alkyl amphopropionates, betaines, alkyl sultaines, alkyl amphoacetates, or a combination of two or more thereof. Preferably, the oral care composition includes an amphoteric surfactant selected from betaine surfactants (also referred to herein as betaines). Examples of betaine surfactants include, e.g., alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine. In some instances, the betaine surfactant is selected from coca betaine, cocamidopropyl betaine, lauryl betaine, laurylhydroxy lauryldimethyl sulfobetaine, betaine, behenyl betaine, capryl/capramidopropyl betaine, lauryl hydroxysultaine, stearyl betaine, and a combination of two or more thereof. For instance, the betaine surfactant may be coco betaine, cocamidopropyl betaine, behenyl betaine, capryl/capramidopropyl betaine, and lauryl betaine, or a combination of two or more thereof. In at least one embodiment, the oral care composition comprises coco betaine, cocamidopropyl betaine, or a combination thereof.

The oral care composition may be formulated to have a weight ratio of the total amount of amino acid derived surfactant to the total amount of amphoteric surfactant of from about 1:1 to about 1:8. In some preferred embodiments, the oral care composition has a weight ratio of the total amount of amino acid derived surfactant to the total amount of amphoteric surfactant from about 1:1 to about 1:8, about 1:1 to about 1:7, about 1:1 to about 1:6, about 1:1 to about 1:5, about 1:1 to about 1:4, about 1:1 to about 1:3, about 1:1 to about 1:2; from about 1:2 to about 1:8, about 1:2 to about 1:7, about 1:2 to about 1:6, about 1:2 to about 1:5, about 1:2 to about 1:4, about 1:2 to about 1:3; from about 1:3 to about 1:8, about 1:3 to about 1:7, about 1:3 to about 1:6, about 1:3 to about 1:5, about 1:3 to about 1:4, or any range or subrange thereof.

The surfactant system may comprise one or more nonionic surfactant(s). The nonionic surfactants may be present in an amount that may vary depending on the other surfactants in the surfactant system or the form of the oral care composition. In some instances, the oral care composition includes one or more nonionic surfactant(s) in an amount of from about 0.3 to about 8 wt. %, based on the total weight of the oral care composition. For example, the amount of nonionic surfactant(s) may be from about 0.3 to about 7 wt. %, about 0.3 to about 6 wt. %, about 0.3 to about 5 wt. %, about 0.3 to about 4 wt. %, about 0.3 to about 3 wt. %, about 0.3 to about 2 wt. %; from about 0.6 to about 8 wt. %, about 0.6 to about 6 wt. %, about 0.6 to about 5 wt. %, about 0.6 to about 4 wt. %, about 0.6 to about 3 wt. %, about 0.6 to about 2 wt. %; from about 0.9 to about 8 wt. %, about 0.9 to about 6 wt. %, about 0.9 to about 5 wt. %, about 0.9 to about 4 wt. %, about 0.9 to about 3 wt. %, about 0.9 to about 2 wt. %; from about 1.2 to about 8 wt. %, about 1.2 to about 6 wt. %, about 1.2 to about 5 wt. %, about 1.2 to about 4 wt. %, about 1.2 to about 3 wt. %, about 1.2 to about 2 wt. %; from about 1.5 to about 8 wt. %, about 1.5 to about 6 wt. %, about 1.5 to about 5 wt. %, about 1.5 to about 4 wt. %, about 1.5 to about 3 wt. %; from about 2 to about 8 wt. %, about 2 to about 6 wt. %, about 2 to about 5 wt. %, about 2 to about 4 wt. %, about 2 to about 3 wt. %; from about 3 to about 8 wt. %, about 3 to about 6 wt. %, about 3 to about 5 wt. %; from about 4 to about 8 wt. %, about 4 to about 6 wt. %, about 4 to about 5 wt. %, or any range or subrange thereof, based on the total weight of the oral care composition.

The one or more nonionic surfactant(s) may be selected from glucosides, compounds produced by the condensation of alkylene oxide groups (hydrophilic in nature) with an organic hydrophobic compound, which may be aliphatic or alkylaromatic in nature. Examples of glucoside surfactants include decyl glucoside, stearyl glucoside, lauryl glucoside, coco-glucoside, cetearyl glucoside, decyl lauryl glucoside, lauroyl ethyl glucoside, myristoyl ethyl glucoside, oleoyl ethyl glucoside, or a combination of two or more thereof. In some embodiments, the surfactant system includes one or more nonionic surfactant(s) chosen from lauryl glucoside, lauroyl ethyl glucoside, myristoyl ethyl glucoside, oleoyl ethyl glucoside, and a combination of two or more thereof. Additionally or alternatively, the glucoside surfactant may be chosen from polyglucosides, such as alkyl polyglucosides.

Further examples of nonionic surfactants include poloxamers, polyethylene oxide condensates of alkyl phenols, products derived from the condensation of ethylene oxide with the reaction product of propylene oxide and ethylene diamine, ethylene oxide condensates of aliphatic alcohols, acids, and esters, long chain tertiary amine oxides, long chain tertiary phosphine oxides, long chain dialkyl sulfoxides and mixtures of such materials. Additional examples of nonionic surfacants include polyoxyethylene, polyoxyethylene sorbitan esters, polyoxyl 40 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, or a combination of two or more thereof. In some instances, the nonionic surfactant comprises amine oxides, fatty acid amides, ethoxylated fatty alcohols, block copolymers of polyethylene glycol and polypropylene glycol, glycerol alkyl esters, polyoxyethytene glycol octylphenol ethers, sorbitan alkyl esters, polyoxyethylene glycol sorbitan alkyl esters, or a combination of two or more thereof.

The surfactant system may comprise one or more anionic surfactant(s) in an amount that may vary depending on the other surfactants in the surfactant system and/or the form of the oral care composition. In some embodiments, the one or more anionic surfactant(s) are present in the oral care composition in an amount of from about 0.3 to about 8 wt. %, based on the total weight of the oral care composition. For example, the amount of anionic surfactant(s) may be from about 0.3 to about 7 wt. %, about 0.3 to about 6 wt. %, about 0.3 to about 5 wt. %, about 0.3 to about 4 wt. %, about 0.3 to about 3 wt. %, about 0.3 to about 2 wt. %; from about 0.6 to about 8 wt. %, about 0.6 to about 6 wt. %, about 0.6 to about 5 wt. %, about 0.6 to about 4 wt. %, about 0.6 to about 3 wt. %, about 0.6 to about 2 wt. %; from about 0.9 to about 8 wt. %, about 0.9 to about 6 wt. %, about 0.9 to about 5 wt. %, about 0.9 to about 4 wt. %, about 0.9 to about 3 wt. %, about 0.9 to about 2 wt. %; from about 1.2 to about 8 wt. %, about 1.2 to about 6 wt. %, about 1.2 to about 5 wt. %, about 1.2 to about 4 wt. %, about 1.2 to about 3 wt. %, about 1.2 to about 2 wt. %; from about 1.5 to about 8 wt. %, about 1.5 to about 6 wt. %, about 1.5 to about 5 wt. %, about 1.5 to about 4 wt. %, about 1.5 to about 3 wt. %; from about 2 to about 8 wt. %, about 2 to about 6 wt. %, about 2 to about 5 wt. %, about 2 to about 4 wt. %, about 2 to about 3 wt. %; from about 3 to about 8 wt. %, about 3 to about 6 wt. %, about 3 to about 5 wt. %; from about 4 to about 8 wt. %, about 4 to about 6 wt. %, about 4 to about 5 wt. %, or any range or subrange thereof, based on the total weight of the oral care composition.

The anionic surfactants may be selected from non-sulfate based surfactants in some cases. For instance, the anionic surfactant(s) may be chosen from sulfonated monoglycerides of fatty acids, acyl isethionates, acyl sarcosinates, acyl taurate, and a combination of two or more thereof. The alkyl groups of the anionic surfactant(s) may comprise a carbon chain of 8 to 21 carbons. In some instance, the alkyl groups of the anionic surfactant(s) comprise a carbon chain of 8 to 19 carbons, 8 to 17 carbons, 8 to 15 carbons, 8 to 13 carbons, 8 to 11 carbons; 9 to 21 carbons, 9 to 19 carbons, 9 to 17 carbons, 9 to 15 carbons, 9 to 13 carbons, 9 to 11 carbons; 11 to 21 carbons, 11 to 19 carbons, 11 to 17 carbons, 11 to 15 carbons, 11 to 13 carbons; 13 to 21 carbons, 13 to 19 carbons, 13 to 17 carbons, or any range or subrange thereof. The anionic surfactants disclosed herein may be incorporated in a salt form. The salt form of the anionic surfactants may have an alkali metal (e.g., sodium or potassium) and/or ammonium group.

Non-limiting examples of acyl isethionates include sodium isethionate, sodium cocoyl isethionate, sodium lauroyl methyl isethionate, sodium cocoyl methyl isethionate, and combinations thereof. Sulfonated monoglycerides of fatty acids include sodium coconut monoglyceride sulfonates and the like. Examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, ammonium lauroyl sarcosinate, and combinations thereof.

The anionic surfactant may be selected from acyl taurates having a structure according to the following formula:

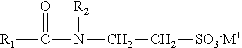

wherein R1 is a saturated or unsaturated, straight or branched alkyl chain with 6 to 18 carbon atoms, R2 is H or methyl, and M⁺is H, sodium, or potassium (e.g., sodium methyl cocoyl taurate).

Additional examples of acyl taurate salts include sodium cocoyl taurate, potassium cocoyl taurate, potassium methyl cocoyl taurate, sodium caproyl methyl taurate, sodium cocoyl taurate, sodium lauroyl taurate, sodium methyl cocoyl taurate (SMCT), sodium methyl lauroyl taurate, sodium methyl myristoyl taurate, sodium methyl oleoyl taurate, sodium methyl palmitoyl taurate, sodium methyl stearoyl taurate, and combinations thereof. In some embodiments, the oral care composition comprises sodium lauroyl methyl taurate (or sodium methyl lauroyl taurate), sodium methyl cocoyl taurate (SMCT), and a combinations thereof.

The oral care composition may have a reduced amount, be substantially free of, or free of sulfate based anionic surfactants. For instance, the oral care composition may have sulfate based anionic surfactants in an amount of about 5 wt. % or less, about 4 wt. % or less, about 3 wt. % or less, about 2 wt. % or less, about 1 wt. % or less, about 0.5 wt. % or less, about 0.1 wt. % or less, or about 0.05 wt. % or less. In some embodiments, the oral care composition is substantially free of or free of sulfate based anionic surfactants. In at least one embodiment, the oral care composition contains 0 wt. % or about 0 wt. % of sulfate based anionic surfactants.

The surfactant system may, in some instances, include one or more cationic surfactant(s) in an amount that may vary depending on the other surfactants in the surfactant system or the form of the oral care composition. In some embodiments, the one or more cationic surfactant(s) are present in the oral care composition in an amount from about 0.3 to about 8 wt. %, based on the total weight of the oral care composition. For example, the amount of cationic surfactant(s) in the oral care composition may be from about 0.3 to about 7 wt. %, about 0.3 to about 6 wt. %, about 0.3 to about 5 wt. %, about 0.3 to about 4 wt. %, about 0.3 to about 3 wt. %, about 0.3 to about 2 wt. %; from about 0.6 to about 8 wt. %, about 0.6 to about 6 wt. %, about 0.6 to about 5 wt. %, about 0.6 to about 4 wt. %, about 0.6 to about 3 wt. %, about 0.6 to about 2 wt. %; from about 0.9 to about 8 wt. %, about 0.9 to about 6 wt. %, about 0.9 to about 5 wt. %, about 0.9 to about 4 wt. %, about 0.9 to about 3 wt. %, about 0.9 to about 2 wt. %; from about 1.2 to about 8 wt. %, about 1.2 to about 6 wt. %, about 1.2 to about 5 wt. %, about 1.2 to about 4 wt. %, about 1.2 to about 3 wt. %, about 1.2 to about 2 wt. %; from about 1.5 to about 8 wt. %, about 1.5 to about 6 wt. %, about 1.5 to about 5 wt. %, about 1.5 to about 4 wt. %, about 1.5 to about 3 wt. %; from about 2 to about 8 wt. %, about 2 to about 6 wt. %, about 2 to about 5 wt. %, about 2 to about 4 wt. %, about 2 to about 3 wt. %; from about 3 to about 8 wt. %, about 3 to about 6 wt. %, about 3 to about 5 wt. %; from about 4 to about 8 wt. %, about 4 to about 6 wt. %, about 4 to about 5 wt. %, or any range or subrange thereof, based on the total weight of the oral care composition.

Examples of cationic surfactants include derivatives of aliphatic quaternary ammonium compounds. The cationic surfactant(s), e.g., aliphatic quaternary ammonium compounds, may have an alkyl group comprising a carbon chain of about 8 to 18 carbon atoms. In some embodiments, the alkyl chain of the cationic surfactant comprises a carbon chain of 8 to 17 carbons, 8 to 15 carbons, 8 to 13 carbons, 8 to 11 carbons; 9 to 18 carbons, 9 to 17 carbons, 9 to 15 carbons, 9 to 13 carbons, 9 to 11 carbons; 11 to 18 carbons, 11 to 17 carbons, 11 to 15 carbons, 11 to 13 carbons; 13 to 18 carbons, 13 to 17 carbons, or any range or subrange thereof. The cationic surfactants disclosed herein may be incorporated in a salt form. The salt form of the cationic surfactants may have comprise a chlorine, amide, amine, dimethylamine, and the like.

The one or more cationic surfactant(s) may comprise lauryl trimethylammonium chloride, cetyl pyridinium chloride, cetyl trimethylammonium bromide, di-isobutylphenoxyethyl-dimethylbenzylammonium chloride, coconut alkyltrimethylammonium nitrite, cetyl pyridinium fluoride, or combinations of two or more thereof. Additional examples of cationic surfactants, such as quaternary ammonium fluorides, are described in U.S. Pat. No. 3,535,421, which is incorporated herein in its entirety by reference for all purposes.

In addition to a surfactant system, the oral care composition typically includes a zinc source (e.g., zinc ion source), preferably in an effective amount. The zinc source (e.g., zinc ion source) may be present in the oral care composition in an amount from about 0.1 to about 8 wt. %, based on the total weight of the oral care composition. For example, the amount of zinc source (e.g., zinc ion source) present in the oral care composition may be from about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.3 to about 8 wt. %, about 0.3 to about 6 wt. %, about 0.3 to about 5 wt. %, about 0.3 to about 4 wt. %, about 0.3 to about 3 wt. %, about 0.3 to about 2 wt. %; from about 0.6 to about 8 wt. %, about 0.6 to about 6 wt. %, about 0.6 to about 5 wt. %, about 0.6 to about 4 wt. %, about 0.6 to about 3 wt. %, about 0.6 to about 2 wt. %; from about 0.9 to about 8 wt. %, about 0.9 to about 6 wt. %, about 0.9 to about 5 wt. %, about 0.9 to about 4 wt. %, about 0.9 to about 3 wt. %, about 0.9 to about 2 wt. %; from about 1.2 to about 8 wt. %, about 1.2 to about 6 wt. %, about 1.2 to about 5 wt. %, about 1.2 to about 4 wt. %, about 1.2 to about 3 wt. %, about 1.2 to about 2 wt. %; from about 1.5 to about 8 wt. %, about 1.5 to about 6 wt. %, about 1.5 to about 5 wt. %, about 1.5 to about 4 wt. %, about 1.5 to about 3 wt. %; from about 2 to about 8 wt. %, about 2 to about 6 wt. %, about 2 to about 5 wt. %, about 2 to about 4 wt. %, about 2 to about 3 wt. %; from about 3 to about 8 wt. %, about 3 to about 6 wt. %, about 3 to about 5 wt. %; from about 4 to about 8 wt. %, about 4 to about 6 wt. %, about 4 to about 5 wt. %, or any range or subrange thereof, based on the total weight of the oral care composition.

The zinc source and/or zinc ion source may be in the form of a salt. For example, the zinc source may comprise one or more zinc salt(s) chosen from zinc sulfate, zinc chloride, zinc acetate, zinc phenol sulfonate, zinc borate, zinc bromide, zinc nitrate, zinc glycerophosphate, zinc benzoate, zinc carbonate, zinc carnosine, zinc citrate, zinc hexafluorosilicate, zinc diacetate trihydrate, zinc oxide, zinc peroxide, zinc salicylate, zinc silicate, zinc stannate, zinc tannate, zinc titanate, zinc tetrafluoroborate, zinc gluconate, zinc lactate, zinc glycinate, zinc phosphate, and a combination of two or more thereof. In some embodiments, the zinc source is selected from zinc citrate, zinc oxide, zinc phosphate, zinc lactate, zinc sulfate, zinc silicate, zinc gluconate, and a combination of two or more thereof. In at least one embodiment, the zinc source consist of zinc citrate, zinc oxide, zinc phosphate, zinc lactate, zinc sulfate, zinc silicate, or a combination of two or more thereof.

The oral care composition may include two or more, three or more, four or more, five or more, or six or more zinc sources. For instance, oral care composition may comprise 2 to 7, 2 to 6, 2 to 5, 2 to 5, or 2 to 4; 3 to 7, 3 to 6, 2 to 5, or 3 to 5 zinc sources. In some embodiments, the oral care composition comprises zinc oxide, zinc citrate, zinc phosphate, or a combination of two or more thereof. In at least one preferred embodiment, the oral care composition comprises zinc phosphate.

The oral care compositions may be formulated to have a weight ratio of the amount of zinc oxide to zinc citrate of from about 1.5:1 to about 4.5:1. For instance, the weight ratio of the amount of zinc oxide to zinc citrate may be from about 1.5:1 to about 4.5:1, about 1.5:1 to about 4:1, about 1.5:1 to about 3.5:1, about 1.5:1 to about 3:1, about 1.5:1 to about 2.5:1; from about 2:1 to about 4.5:1, about 2:1 to about 4:1, about 2:1 to about 3.5:1, about 2:1 to about 3:1; from about 2.5:1 to about 4.5:1, about 2.5:1 to about 4:1, about 2.5:1 to about 3.5:1; from about 3:1 to about 4.5:1, about 3:1 to about 4:1, or any range or subrange formed thereof. In some embodiments, the weight ratio of the amount of zinc oxide to zinc citrate is about 2:1, about 2.5:1, about 3:1, about 3.5:1, about 4:1, or a range formed thereof.

The oral care compositions typically comprise a stannous source (e.g., stannous ion source), preferably in an effective amount. The stannous source (e.g., stannous ion source) may be present in the oral care composition in an amount from about 0.1 to about 5 wt. %, based on the total weight of the oral care composition. For example, the amount of stannous source (e.g., stannous ion source) present in the oral care composition may be from about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 4 to about 5 wt. %, or any range or subrange thereof, based on the total weight of the oral care composition.

The stannous source (e.g., stannous ion source) may be selected from the group consisting of: stannous fluoride, stannous chloride, stannous pyrophosphate, stannous formate, stannous acetate, stannous gluconate, stannous lactate, stannous tartrate, stannous oxalate, stannous malonate, stannous citrate, stannous ethylene glyoxide, and combinations of two or more thereof. The stannous source may comprise stannous fluoride, stannous chloride, stannous acetate, or a combination of two or more thereof. In at least one embodiment, the stannous source comprises stannous fluoride.

The oral care composition may include two or more, three or more, four or more, five or more, or six or more of stannous sources (e.g., stannous ion sources). For instance, oral care composition may comprise 2 to 7, 2 to 6, 2 to 5, 2 to 5, or 2 to 4; 3 to 7, 3 to 6, 2 to 5, or 3 to 5 stannous sources. In some embodiments, the oral care composition comprises stannous fluoride and stannous pyrophosphate. Additionally or alternatively, the oral care composition may comprise stannous fluoride and stannous chloride.

The oral care compositions may include a fluoride ion source. The fluoride ion source may be present in an effective amount. In some cases, the fluoride ion source is present in the oral care composition in an amount of about 0.01 to about 5 wt. %, based on the total weight of the oral care composition. For example, the fluoride ion source is present in an amount of about 0.01 to about 4 wt. %, about 0.01 to about 3 wt. %, about 0.01 to about 2 wt. %, about 0.01 to about 1 wt. %; from about 0.05 to about 5 wt. %, about 0.05 to about 4 wt. %, about 0.05 to about 3 wt. %, about 0.05 to about 2 wt. %, about 0.05 to about 1 wt. %; from about 0.1 to about 5 wt. %, about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 4 to about 5 wt. %, or any range or subrange thereof, based on the total weight of the oral care composition.

The fluoride ion source may be selected from soluble fluoride ion salts. For example, the fluoride ion source may comprise sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluorides, or a combination of two or more thereof. In some embodiments, the fluoride ion source comprises sodium fluoride, sodium monofluorophosphate, or a combination thereof. Additional examples of fluoride ion source are disclosed in U.S. Pat. Nos. 3,535,421, 4,885,155, and 3,678,154, the disclosure of each of which is hereby incorporated by reference in their entirety.

In certain embodiments, the oral care composition of the disclosure may contain stannous fluoride and a source of fluoride ions or fluorine-providing agents in amounts sufficient to supply, in total, from 25 ppm to 25,000 ppm (mass fraction) of fluoride ions, generally at least 500 ppm, e.g., from about 500 to about 2000 ppm, from about 800 to about 1800 ppm, from about 1000 to about 1600 ppm, from about 1200 to about 1550 ppm, or about 1450 ppm.

The oral care compositions may include one or more abrasive(s), e.g., in an amount from about 5 to about 35 wt. %, based on the total weight of the oral care composition. In some embodiments, the one or more abrasive(s) is present in an amount from about 5 to about 30 wt. %, about 5 to about 25 wt. %, about 5 to about 21 wt. %, about 5 to about 17 wt. %, about 5 to about 14 wt. %, about 5 to about 11 wt. %; from about 10 to about 35 wt. %, about 10 to about 30 wt. %, about 10 to about 25 wt. %, about 10 to about 21 wt. %, about 10 to about 17 wt. %, about 10 to about 14 wt. %; from about 15 to about 35 wt. %, about 15 to about 30 wt. %, about 15 to about 25 wt. %, about 15 to about 21 wt. %, about 15 to about 19 wt. %; from about 18 to about 35 wt. %, about 18 to about 30 wt. %, about 18 to about 25 wt. %, about 18 to about 21 wt. %; from about 21 to about 35 wt. %, about 21 to about 30 wt. %, about 21 to about 25 wt. %; from about 24 to about 35 wt. %, about 24 to about 30 wt. %; from about 27 to about 35 wt. %, about 27 to about 30 wt. %, or any range or subrange thereof, based on the total weight of the oral care composition.

The one or more abrasive(s) may include: silica, silicate, silicon, alumina (including calcined aluminum oxide), aluminosilicates, such as bentonite, zeolite, kaolin, and mica, siliceous or diatomaceous earth, pumice, calcium carbonate, cuttlebone, insoluble phosphates, composite resins, such as melamine resin, phenolic resin, urea-formaldehyde resin, polycarbonate, silicon carbide, boron carbide, microcrystalline wax, microcrystalline cellulose, including combinations of colloidal microcrystalline cellulose and carboxymethylcellulose, derivatives thereof, or combinations of two or more thereof. For example, the one or more abrasive may be selected from silicas including gels and precipitates, calcium carbonate, dicalcium orthophosphate dihydrate, calcium pyrophosphate, tricalcium phosphate, calcium polymetaphosphate, insoluble sodium polymetaphosphate, hydrated alumina, resinous abrasive materials such as particulate condensation products of urea and formaldehyde, and combinations thereof.

As used herein, “mica” refers to any of a group of hydrous aluminum silicate minerals with plate morphology and/or perfect basal (micaceous) cleavage. Mica can be, for example, sheet mica, scrap mica or flake mica, as exemplified by muscovite, biotite or phlogopite type micas. The abrasive may be selected from insoluble phosphates, such as orthophosphates, polymetaphosphates, pyrophosphates, and a combination thereof. Examples of phosphates include dicalcium orthophosphate dihydrate, dicalcium phosphate dihydrate, calcium hydrogen phosphate, calcium pyrophosphate, p-calcium pyrophosphate, tricalcium phosphate, calcium metaphosphate, potassium metaphosphate, sodium metaphosphate, or a combination thereof.

Synthetic silicas include both silica gels and precipitated silicas that are prepared by the neutralization of aqueous silicate solutions with a strong mineral acid. Abrasives comprising silica may be particularly useful in certain embodiments of the oral care composition. In certain embodiments, the oral compositions may comprise a particularly efficacious combination of silica abrasive particle species, the abrasive may be selected from high cleaning silica.

The oral care compositions may include an abrasive system comprising two or more abrasives. For example, an abrasive system comprise 2 to 7, 2 to 6, 2 to 5, 2 to 5, or 2 to 4; 3 to 7, 3 to 6, 2 to 5, or 3 to 5 abrasives. The abrasives may comprise one or more cleaning abrasive and/or one or more polishing abrasives. As appreciated by one of skill in the art, a single abrasive species typically performs at least some cleaning and polishing simultaneously. However, particles are generally categorized in the art by the predominant effect they have on a target oral surface. Typically, “polishing abrasives” are considered to be relatively small particles having high hardness, where abrasives with relatively large particle sizes and low hardness are considered to be “cleaning abrasives.” In certain embodiments, the oral care composition comprises two or more abrasives comprising silica. In some embodiments, the first abrasive is selected to be a harder and smaller abrasive, e.g., a higher cleaning and/or polishing abrasive, and the second abrasive is a typical cleaning abrasive. In some embodiments, the oral care composition includes at least one polishing abrasive and/or at least one cleaning abrasive. Further examples of abrasives are disclosed in U.S. Patent Publication No. 2007/140986, which is incorporated herein in its entirety by reference for all purposes.

The abrasive(s) may have at least one of the following properties: N₂ BET surface area of less than 50 m²/g, an Einlehner hardness of from 4 to 11, an oil absorption of from 80 cc/100 g to 100 cc/100 g, or a combination of two or more thereof. The abrasive may have a N₂ BET surface area of from 1 m²/g to 50 m²/g. e.g., from 1 m²/g to 45, from 1 m²/g to 40 m²/g, from 1 m²/g to 35 m²/g, or from 1 m²/g to 30 m²/g. The abrasive may have an oil absorption of from 60 cc/100 g to 120 cc/100 g. from 70 cc/100 g to 110 cc/100 g, or from 80 cc/100 g to 100 cc/100 g, e.g., linseed oil absorption. Oil absorption may be determined by various means known by those of skill in the art. For example, oil absorption may be determined by absorption of linseed oil or dibutyl phthalate (DBP) per 100 grams or abrasive. Oil absorption values can be measured using the ASTM Rub-Out Method D281. In at least one embodiment, the one or more abrasive(s) comprises an abrasive (e.g. a silica abrasive) having have N₂ BET surface area of less than 50 m²/g and an Einlehner hardness of from 4 to 11, and an oil absorption of from 80 cc/100 g to 100 cc/100 g.

The oral care composition may include one or more thickening agent(s), e.g., in an amount from about 0.5 to about 20 wt. %, based on the total weight of the oral care composition. For example, the amount of thickening agent(s) in the oral care composition may be from about 0.5 to about 17 wt. %, about 0.5 to about 14 wt. %, about 0.5 to about 11 wt. %, about 0.5 to about 9 wt. %, about 0.5 to about 7 wt. %, about 0.5 to about 5 wt. %, about 0.5 to about 4 wt. %, about 0.5 to about 3 wt. %; from about 1 to about 20 wt. %, about 1 to about 17 wt. %, about 1 to about 14 wt. %, about 1 to about 11 wt. %, about 1 to about 9 wt. %, about 1 to about 7 wt. %, about 1 to about 5 wt. %, about 1 to about 4 wt. %, about 1 to about 3 wt. %; from about 2 to about 20 wt. %, about 2 to about 17 wt. %, about 2 to about 14 wt. %, about 2 to about 11 wt. %, about 2 to about 9 wt. %, about 2 to about 7 wt. %, about 2 to about 5 wt. %, about 2 to about 4 wt. %, about 2 to about 3 wt. %; from about 4 to about 20 wt. %, about 4 to about 17 wt. %, about 4 to about 14 wt. %, about 4 to about 11 wt. %, about 4 to about 9 wt. %, about 4 to about 7 wt. %; from about 7 to about 20 wt. %, about 7 to about 17 wt. %, about 7 to about 14 wt. %, about 7 to about 11 wt. %; from about 10 to about 20 wt. %, about 10 to about 17 wt. %, about 10 to about 14 wt. %; from about 14 to about 20 wt. %, about 14 to about 17 wt. %, or any range or subrange thereof, based on the total weight of the oral care composition.

Thickening agents may be referred to as “thickeners” or “viscosity modifying agents.” Thickening agents are typically included to increase the viscosity of the oral care compositions. Nonetheless, in some instances, certain thickening agents provide additional, surprising benefits to the oral care compositions.

Examples of thickening agent(s), which may be included or excluded from certain embodiments, include those selected from a polysaccharide, a silica thickener, an acrylate, a polymer, and a combination of two or more thereof. Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, carrageenan or a combination of two more thereof. Natural gums such as karaya, gum arabic, and gum tragacanth can also be incorporated. Colloidal magnesium aluminum silicate can also be used as a component of the thickening composition to further improve the composition's texture. Silica thickeners, which form polymeric structures or gels in aqueous media, may be present. Silica thickeners are typically physically and functionally distinct from the particulate silica abrasives as the silica thickeners are very finely divided and provide little or no abrasive action.

In some embodiments, the one or more thickening agent(s) comprises a polymer selected from polysaccharides, acrylates, polyvinyl pyrrolidone, polyitaconates, an acrylamides, and combinations thereof. Polyvinyl pyrrolidone generally refers to a polymer containing vinylpyrrolidone (e.g., N-vinylpyrrolidone, N-vinyl-2-pyrrolidione, and N-vinyl-2-pyrrolidinone) as a monomeric unit. The monomeric unit may include a polar imide group, four non-polar methylene groups, and a non-polar methane group. The polyvinyl pyrrolidone may have an average molecular weight in the range 5,000 to 100,000, preferably in the range 5,000 to 50,000. Polyvinyl pyrrolidones that have average molecular weights of 10,000, 30,000 and 40,000 may be commercially available from Sigma Chemical Co., GAF Corporation and Sigma Chemical Co. The polyvinyl pyrrolidone may form a hydrogen peroxide-polyvinyl pyrrolidone polymer complexes. Examples of polyvinyl pyrrolidone complexes include those disclosed in U.S. Pat. No. 5,122,370, the contents of which are incorporated herein by reference. In some embodiments, the polymer comprises crosslinked polyvinyl pyrrolidone (crosslinked PVP). In other embodiments, however, the oral care composition is substantially free of or free of polyvinyl pyrrolidone.

Examples of acrylates that may be included in the oral care composition include, e.g., isobutyl acrylate, tert-butyl acrylate, 2-ethylhexyl acrylate, lauryl acrylate, lauryl/tridecyl acrylate, cetyl acrylate, stearyl acrylate, cyclohexyl acrylate, benzyl acrylate, isobornyl acrylate, 2-methoxyethyl acrylate, 2-ethoxyethyl acrylate, 2-ethoxyethoxyethyl acrylate, 2-phenoxyethyl acrylate, tetrahydrofurfuryl acrylate, 2-hydroxyethyl acrylate, 2-hydroxypropyl acrylate, 4-hydroxybutyl acrylate, dimethylaminoethyl acrylate, 1,4-butanediol acrylate, or a combination of two or more thereof.

The acrylate may be chosen from diacrylates. In some embodiments, the oral care composition includes a diacrylate chosen from 1,4-butanediol, 1,6-hexanediol, tetraethylene glycol, tripropylene glycol, ethoxylated bisphenol-A, and a combination of two or more thereof. Triacrylate monomers include trimethylol propane, ethoxylated, glyceryl propoxy, and pentaerythritol.

Acrylates further include methacrylates, such as methyl methacrylate, ethyl methacrylate, n-butyl methacrylate, isobutyl methacrylate, tert-butyl methacrylate, 2-ethylhexyl methacrylate, lauryl methacrylate, alkyl methacrylate, tridecyl methacrylate, stearyl methacrylate, cyclohexyl methacrylate, benzyl methacrylate, isobornyl methacrylate, 2-hydroxyethyl methacrylate, 2-hydroxypropyl methacrylate, dimethylaminoethyl methacrylate, diethylaminoethyl methacrylate, glycidyl methacrylate, tetrahydrofurfuryl methacrylate, allyl methacrylate, ethylene glycol methacrylate, triethylene glycol methacrylate, tetraethylene glycol methacrylate, 1,3-butyleneglycol methacrylate, 1,6-hexanediol methacrylate, trimethylopropane methacrylate, ethoxyethyl methacrylate, trifluoroethyl methacrylate, or a combination of two or more thereof.

Examples of acrylamides include, but are not limited to, acrylamide, methacrylamide and di(C₁-C₃₀) alkyl-acrylamides and -methacrylamides such as those of methyl, ethyl, propyl, butyl, pentyl, hexyl and the like. N-substituted acrylamides that may be suitable include N-ethylacrylamide, N-tert-butylacrylamide, N-tert-octylacrylamide, N-octylacrylamide, N-decylacrylamide, N-dodecylacrylamide and the corresponding N-substituted methacrylamides. Other N-substituted acrylamides include N-hydroxymethyl acrylamide, N-isopropylacrylamide, N-methylacrylamide, N,N′-methylenebisacrylamide, N-isobutoxymethylacrylamide, N,N-dimethylacrylamide, 2-acrylamido-2-methylpropanesulfonic acid, or a combination of two or more thereof.

The oral care compositions may include one or more polyol(s). The one or more polyol(s) may be present in the oral care composition in an amount from about 10 to about 50 wt. %, based on the total weight of the oral care composition. For example, the oral care composition may include polyol(s) in an amount from about 10 to about 50 wt. %, about 10 to about 45 wt. %, about 10 to about 40 wt. %, about 10 to about 37 wt. %, about 10 to about 34 wt. %, about 10 to about 31 wt. %, about 10 to about 28 wt. %, about 10 to about 25 wt. %; from about 15 to about 50 wt. %, about 15 to about 45 wt. %, about 15 to about 40 wt. %, about 15 to about 37 wt. %, about 15 to about 34 wt. %, about 15 to about 31 wt. %, about 15 to about 28 wt. %, about 15 to about 25 wt. %; from about 20 to about 50 wt. %, about 20 to about 45 wt. %, about 20 to about 40 wt. %, about 20 to about 37 wt. %, about 20 to about 34 wt. %, about 20 to about 31 wt. %, about 20 to about 28 wt. %; from about 25 to about 50 wt. %, about 25 to about 45 wt. %, about 25 to about 40 wt. %, about 25 to about 37 wt. %, about 25 to about 34 wt. %, about 25 to about 31 wt. %; from about 28 to about 50 wt. %, about 28 to about 45 wt. %, about 28 to about 40 wt. %, about 28 to about 37 wt. %, about 28 to about 34 wt. %, about 28 to about 31 wt. %; from about 31 to about 50 wt. %, about 31 to about 45 wt. %, about 31 to about 40 wt. %, about 31 to about 37 wt. %; from about 34 to about 50 wt. %, about 34 to about 45 wt. %, about 34 to about 40 wt. %; from about 37 to about 50 wt. %, about 37 to about 45 wt. %, or any range or subrange thereof, based on the total weight of the oral care composition.

The polyol(s) may be chosen from glycols or compounds with numerous hydroxyl groups. The one or more polyols may be liquid at ambient temperature (25° C.). The polyol may be a humectant. In some preferred embodiments, the polyol(s) comprises glycerin, glycol, inositol, maltitol, mannitol, sorbitol, xylitol, propylene glycol, polypropylene glycol (PPG), polyethylene glycol (PEG), a block copolymer of PPG and PEG, a saccharide (e.g., fructose, glucose, sucrose and mixtures of saccharides, such as honey), or a combination of two or more thereof. For instance, the oral care composition comprises maltitol, mannitol, sorbitol, xylitol, a polypropylene glycol (PPG), a polyethylene glycol (PEG), a block copolymer of PPG and PEG, or a combination or two or more thereof.

In some cases, the oral care composition includes one or more polyol selected from the group consisting of C₂-C₃₂ polyols. The one or more polyols may have from 2 to 32 carbon atoms, from 3 to 16 carbon atoms, or from 3 to 12 carbon atoms. For example, the oral care composition may comprise ethylene glycol, propylene glycol, butylene glycol, hexylene glycol, glycerin, diglycerin, diethylene glycol, dipropylene glycol, or a combination of two or more thereof. Additional, non-limiting examples of polyols that may, optionally, be included in the oral care include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers, such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture of two or more thereof.

Additionally or alternatively, the oral care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof. In some embodiments, the polyol is a polyethylene glycol, such as polyethylene glycol 600 (CAS-25322-68-3). In some embodiments, the compositions of the disclosure comprise one or more polyethylene glycols, for example, polyethylene glycols in a molecular weight range from 200 to 800. For example, the compositions may comprise one or more of polyethylene glycol 200, polyethylene glycol 300, polyethylene glycol 400, polyethylene glycol, 600 or polyethylene glycol 800

The oral care compositions may include water in some embodiments. The water may be present in the oral care composition in an amount from about 10 to about 50 wt. %, based on the total weight of the oral care composition. For example, the oral care composition may include water in an amount from about 10 to about 50 wt. %, about 10 to about 45 wt. %, about 10 to about 40 wt. %, about 10 to about 37 wt. %, about 10 to about 34 wt. %, about 10 to about 31 wt. %, about 10 to about 28 wt. %, about 10 to about 25 wt. %; from about 15 to about 50 wt. %, about 15 to about 45 wt. %, about 15 to about 40 wt. %, about 15 to about 37 wt. %, about 15 to about 34 wt. %, about 15 to about 31 wt. %, about 15 to about 28 wt. %, about 15 to about 25 wt. %; from about 20 to about 50 wt. %, about 20 to about 45 wt. %, about 20 to about 40 wt. %, about 20 to about 37 wt. %, about 20 to about 34 wt. %, about 20 to about 31 wt. %, about 20 to about 28 wt. %; from about 25 to about 50 wt. %, about 25 to about 45 wt. %, about 25 to about 40 wt. %, about 25 to about 37 wt. %, about 25 to about 34 wt. %, about 25 to about 31 wt. %; from about 28 to about 50 wt. %, about 28 to about 45 wt. %, about 28 to about 40 wt. %, about 28 to about 37 wt. %, about 28 to about 34 wt. %, about 28 to about 31 wt. %; from about 31 to about 50 wt. %, about 31 to about 45 wt. %, about 31 to about 40 wt. %, about 31 to about 37 wt. %; from about 34 to about 50 wt. %, about 34 to about 45 wt. %, about 34 to about 40 wt. %; from about 37 to about 50 wt. %, about 37 to about 45 wt. %, or any range or subrange thereof, based on the total weight of the oral care composition.

The oral care compositions may, in some cases, comprise one or more amino acid(s) in an effective amount. In some embodiments, the one or more amino acid(s) may be present in the oral care composition in an amount from about 0.1 to about 10 wt. %, about 0.1 to about 8 wt. %, about 0.1 to about 6 wt. %, about 0.1 to about 4 wt. %, about 0.1 to about 2 wt. %, about 0.1 to about 1 wt. %, about 0.1 to about 0.5 wt. %; from about 0.5 to about 10 wt. %, about 0.5 to about 8 wt. %, about 0.5 to about 6 wt. %, about 0.5 to about 4 wt. %, about 0.5 to about 2 wt. %, about 0.5 to about 1 wt. %; from about 1 to about 10 wt. %, about 1 to about 8 wt. %, about 1 to about 6 wt. %, about 1 to about 4 wt. %, about 1 to about 2 wt. %; from about 2 to about 10 wt. %, about 2 to about 8 wt. %, about 2 to about 6 wt. %, about 2 to about 4 wt. %; from about 3 to about 10 wt. %, about 3 to about 8 wt. %, about 3 to about 6 wt. %, about 3 to about 4 wt. %; from about 4 to about 10 wt. %, about 4 to about 8 wt. %, about 4 to about 6 wt. %; from about 6 to about 10 wt. %, about 6 to about 8 wt. %; from about 8 to about 10 wt. %, including any range or subrange thereof, based on the total weight of the oral care composition.

The one or more amino acids may be selected from basic amino acids, neutral amino acids, and combinations thereof. The basic amino acids may be selected from naturally occurring basic amino acids, such as arginine, lysine, and histidine, and non-naturally occurring basic amino acids having a carboxyl group and an amino group in the molecule, which are water-soluble and provide an aqueous solution with a pH of 7 or greater. Examples of basic amino acids include arginine, lysine, serine, citrulline, ornithine, creatine, histidine, diaminobutanoic acid, diaminoproprionic acid, salts thereof or combinations thereof. In some embodiments, the basic amino acids are selected from arginine, citrulline, and ornithine. In certain embodiments, the basic amino acid is arginine, e.g., L-arginine, or a salt thereof. In at least one embodiment, the total amount of amino acids consists of arginine and/or lysine. Additionally or alternatively, the one or more amino acid can be chosen from neutral amino acids, which can include, but are not limited to, one or more neutral amino acids selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof.

In some embodiments, the oral care compositions may include one or more sweeteners.

The oral care composition may include caloric sweeteners and/or non-caloric sweeteners. Examples of non-caloric sweeteners include saccharin, for example, sodium saccharin, acesulfame, neotame, cyclamate or sucralose; natural high-intensity sweeteners, such as thaumatin, stevioside or glycyrrhizin; or sugar alcohols, such as sorbitol, xylitol, maltitol and mannitol. Examples of caloric sweeteners include sugars, such as fructose, glucose, sucrose, and high fructose syrups.

The one or more sweetener(s) may be present in the oral care composition in an amount from about 0.1 to about 50 wt. %, based on the total weight of the oral care composition. For example, the oral care composition may have a total amount of sweetener(s) from about 0.1 to about 40 wt. %, about 0.1 to about 30 wt. %, about 0.1 to about 20 wt. %, about 0.1 to about 10 wt. %, about 0.1 to about 5 wt. %, about 0.1 to about 3 wt. %; from about 1 to about 50 wt. %, about 1 to about 40 wt. %, about 1 to about 30 wt. %, about 1 to about 20 wt. %, about 1 to about 10 wt. %, about 1 to about 5 wt. %, about 1 to about 3 wt. %; from about 5 to about 50 wt. %, about 5 to about 40 wt. %, about 5 to about 30 wt. %, about 5 to about 20 wt. %, about 5 to about 10 wt. %; from about 10 to about 50 wt. %, about 10 to about 45 wt. %, about 10 to about 40 wt. %, about 10 to about 35 wt. %, about 10 to about 30 wt. %, about 10 to about 25 wt. %; from about 15 to about 50 wt. %, about 15 to about 45 wt. %, about 15 to about 40 wt. %, about 15 to about 37 wt. %, about 15 to about 34 wt. %, about 15 to about 31 wt. %, about 15 to about 28 wt. %, about 15 to about 25 wt. %; from about 20 to about 50 wt. %, about 20 to about 45 wt. %, about 20 to about 40 wt. %, about 20 to about 37 wt. %, about 20 to about 34 wt. %, about 20 to about 31 wt. %, about 20 to about 28 wt. %; from about 25 to about 50 wt. %, about 25 to about 45 wt. %, about 25 to about 40 wt. %, about 25 to about 37 wt. %, about 25 to about 34 wt. %, about 25 to about 31 wt. %; from about 28 to about 50 wt. %, about 28 to about 45 wt. %, about 28 to about 40 wt. %, about 28 to about 37 wt. %, about 28 to about 34 wt. %, about 28 to about 31 wt. %; from about 31 to about 50 wt. %, about 31 to about 45 wt. %, about 31 to about 40 wt. %, about 31 to about 37 wt. %; from about 34 to about 50 wt. %, about 34 to about 45 wt. %, about 34 to about 40 wt. %; from about 37 to about 50 wt. %, about 37 to about 45 wt. %, or any range or subrange thereof, based on the total weight of the oral care composition.

In some embodiments, the oral care composition preferably is substantially free or free of caloric sweeteners. For example, the oral care compositions may have about 4 wt. % or less, about 3 wt. % or less, about 2 wt. % or less, about 1 wt. % or less, about 0.5 wt. % or less, or about 0.1 wt. % or less, based on the weight of the oral care composition. In at least one embodiment, the oral care composition contains about 0 wt. % or 0 wt. % of caloric sweeteners, based on the weight of the oral care composition.

The oral care compositions of the present disclosure may include a flavoring agent. The flavoring agent is typically incorporated in the oral care composition at a concentration of about 0.01 to about 3 wt. % by weight of the oral care composition. For example, the amount of flavoring agent(s) present in the oral care composition may be from about 0.01 to about 2 wt. %, about 0.01 to about 1 wt. %, about 0.01 to about 0.5 wt. %, about 0.01 to about 0.1 wt. %; from about 0.1 to about 3 wt. %, about 0.1 to about 2 wt. %, about 0.1 to about 1 wt. %, about 0.1 to about 0.5 wt. %; from about 0.5 to about 3 wt. %, about 0.5 to about 2 wt. %, about 0.5 to about 1 wt. %; from about 1 to about 3 wt. %, about 1 to about 2 wt. %; from about 2 to about 3 wt. %, including any range or subrange thereof, based on the total weight of the oral care composition.

Suitable flavoring agents include, but are not limited to, essential oils and various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Additional flavoring agents may include, but are not limited to menthol, artificial vanilla, cinnamon derivatives, and various fruit flavors, spearmint oil, peppermint oil, cinnamon oil, oil of wintergreen (methylsalicylate), clove oil, bay oil, anise oil, eucalyptus oil, thyme oil, cedar leaf oil, oil of nutmeg, oil of sage, oil of bitter almonds, cassia oil, and a combination of two or more thereof.

The oral care compositions may include one or more pigments, such as whitening pigments, and/or colorants. In some embodiments, the whitening pigments include particles ranging in size from about 0.1 μm to about 10 μm with a refractive index greater than about 1.2. Suitable whitening pigments include, without limitation, titanium dioxide particles, zinc oxide particles, aluminum oxide particles, tin oxide particles, calcium oxide particles, magnesium oxide particles, barium oxide particles, silica particles, zirconium silicate particles, mica particles, talc particles, tetracalcium phosphate particles, amorphous calcium phosphate particles, alpha-tricalcium phosphate particles, beta-tricalcium phosphate particles, hydroxyapatite particles, calcium carbonate particles, zinc phosphate particles, silicon dioxide particles, zirconium silicate particles, or the like, or mixtures and combinations thereof.

Exemplary colorants can include natural or uncertified colors from natural sources or certified colors for the effect of color. In some embodiments, the colorant can include dyes, certified aluminum lakes or colors derived from a natural source. The colorant may be water-based, oil-based or dry. The colorants can be primary colors, blends of colors or discrete mixtures of colors, such as confetti. The concentrations of the colorant in the oral care composition may be from trace amount to about 0.6 wt. %, from about 0.1 to about 0.5 wt. %, about 0.2 to about 0.4 wt. %, or about 0.15 to about 0.35 wt. %, based on the total weight of the oral care composition.

The oral care composition may include one or more pH adjusters to increase or decrease the overall pH of the oral care composition. For example, one or more acids may be included to decrease the pH of the oral care composition. Examples of suitable acids for decreasing the pH of the oral care composition include, but are not limited to, citric acid, acetic acid, and the like. The oral care composition may include one or more bases, such as sodium hydroxide, potassium hydroxide and the like, to increase the pH of the oral care composition. Additional or alternative acids and bases that are suitable for adjusting the pH of the oral care composition are readily known to one of ordinary skill in the art.

The amount of the pH adjuster in the oral care composition may be based on the desired pH of the final oral care composition and/or product. For example, the total amount of the pH adjuster may range from about 0.05 to about 20 wt. %, based on the total weight of the oral care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the oral care composition.

The oral care compositions may have a pH from 4.5 to about 10, 4.5 to about 9, 4.5 to about 8, 4.5 to about 7, 4.5 to about 6; from about 5 to about 10, about 5 to about 9, about 5 to about 8, about 5 to about 7, about 5 to about 6; from about 6 to about 10, about 6 to about 9, about 6 to about 8, or about 6 to about 7; from about 7 to about 10, about 7 to about 9, or about 7 to about 8, including any ranges and subranges therebetween.

In some embodiments, the oral care compositions are either essentially free of, free of or do not include any complexing agents for increasing solubility of zinc phosphate. In some embodiments, the oral care composition may comprise about 4 wt. % or less, about 3 wt. % or less, about 2 wt. % or less, about 1 wt. % or less, about 0.5 wt. % or less, about 0.1 wt. % or less of complexing agents for increasing solubility of zinc phosphate. Examples of known complexing agents that can be excluded from the compositions of the present disclosure include the chelating agents taught in U.S. Patent Application No. 2007/0025928, the disclosure of which is hereby incorporated by reference in its entirety. Such chelating agents include mineral surface-active agents, including mineral surface-active agents that are polymeric and/or polyelectrolytes and that are selected from phosphorylated polymers, wherein if the phosphorylated polymer is a polyphosphate, the polyphosphate has average chain length of 3.5 or more, such as 4 or more; polyphosphonates; polycarboxylates; carboxy-substituted polymers; copolymers of phosphate- or phosphonate-containing monomers or polymers with ethylenically unsaturated monomers, amino acids, proteins, polypeptides, polysaccharides, poly(acrylate), poly(acrylamide), poly(methacrylate), poly(ethacrylate), poly(hydroxyalkylmethacrylate), poly(vinyl alcohol), poly(maleic anhydride), poly(maleate) poly(amide), poly(ethylene amine), poly(ethylene glycol), poly(propylene glycol), poly(vinyl acetate) and poly(vinyl benzyl chloride); and mixtures thereof. Other known complexing agents that can be excluded from the compositions of the present disclosure include those taught in CA 2634758, the disclosure of which is incorporated here by reference in its entirety. Examples include polyphosphorylated inositol compounds such as phytic acid, myo-inositol pentakis(dihydrogen phosphate); myo-inositol tetrakis(dihydrogen phosphate), myo-inositol trikis(dihydrogen phosphate), and alkali metal, alkaline earth metal or ammonium salts of any of the above inositol compounds. Phytic acid is also known as myo-inositol 1,2,3,4,5,6-hexakis (dihydrogen phosphate) or inositol hexaphosphoric acid.

In another aspect, the present disclosure provides a method of treatment or prevention of erosive tooth demineralization, repair of enamel, gingivitis, plaque, and/or dental caries, the method comprising the application to the oral cavity of a subject in need thereof an oral composition disclosed herein, e.g., by brushing, for example, one or more times per day. The methods may treat, reduce or control the incidence of enamel erosion.

The methods typically comprise applying any of the compositions as described herein to the teeth, e.g., by brushing, rubbing, swishing when the oral care composition is in the form of a mouthwash, or otherwise administering the oral care compositions to the oral cavity of a subject. The oral care compositions can be administered regularly, such as, for example, one or more times per day. In various embodiments, administering the oral care compositions of the present disclosure to a subject can provide one or more of the following benefits: (i) reduce hypersensitivity of the teeth, (ii) reduce plaque accumulation, (iii) reduce or inhibit demineralization and promote remineralization of the teeth, (iv) inhibit microbial biofilm formation in the oral cavity, (v) reduce or inhibit gingivitis, (vi) promote healing of sores or cuts in the mouth, (vii) reduce levels of acid producing bacteria, (viii) increase relative levels of non-cariogenic and/or non-plaque forming bacteria, (ix) reduce or inhibit formation of dental caries, (x) reduce, repair or inhibit pre-carious lesions of the enamel, e.g., as detected by quantitative light-induced fluorescence (QLF) or electrical caries measurement (ECM), (xi) treat, relieve or reduce dry mouth, (xii) clean the teeth and oral cavity, (xiii) reduce erosion, (xiv) whiten teeth; (xv) reduce tartar build-up, and/or (xvi) promote systemic health, including cardiovascular health, e.g., by reducing potential for systemic infection via the oral tissues. The disclosure further provides compositions for use in any of the above methods. Further embodiments provide methods wherein at least one tooth is remineralized after administration of an oral composition as described herein.

### EXAMPLES

### Example 1

Non-limiting, examples of oral care compositions (Ex. A-C) were prepared in accordance with aspects of the invention. A comparative composition (Comp. 1) was also prepared. Example Compositions A-C and Comparative Composition 1 were in the form of a dentifrice and had the formulations shown in Table 1.

TABLE 1

| | Comp. 1 | Ex. A | Ex. B | Ex. C |
|---|---|---|---|---|
| US INCI Name | (wt. %) | (wt. %) | (wt. %) | (wt. %) |
| Sorbitol | 27 | 27 | 27 | 27 |
| Glycerin | 6 | 6 | 6 | 6 |
| Xylitol | | 5 | | |
| Synthetic Abrasive Silica | 20 | 20 | 20 | 20 |
| Polyethylene Glycol 600 | 2 | | 2 | 2 |
| Tetrasodium Pyrophosphate | 2 | 2 | 2 | 2.5 |
| Trisodium Citrate Dihydrate | 1 | 1 | 1 | 1 |
| Zinc Phosphate Hydrate | 1 | 1 | 1 | 1 |
| Sodium Carboxymethyl | 1.8 | 0.8 | 1.8 | 1.8 |
| Cellulose (microcrystalline | | | | |
| and/or non-microcrystalline) | | | | |
| Citric Acid | 0.2 | 0.2 | 0.2 | 0.2 |
| Titanium Dioxide | 0.5 | | 0.5 | 0.5 |
| Stannous Fluoride | 0.5 | 0.5 | 0.5 | 0.5 |
| Xanthan Gum and Synthetic | 1.8 | 1.8 | 1.8 | 1.8 |
| Thickening Silica | | | | |
| Sodium Lauryl Sulfate | 1.5 | | | |
| Cocamidopropyl Betaine | 0.4 | 0.4 | 0.9 | 0.9 |
| Sodium Cocoyl Glutamate | | 0.22 | 0.47 | 0.47 |
| Alkyl Polyglucoside | | | 0.77 | 0.77 |
| Poloxomer | | | | 0.5 |
| Flavoring/Sweetening Agents | 1.7 | 1.6 | 1.7 | 1.7 |
| (e.g., flavoring agents, | | | | |
| saccharin, sucralose, and/or | | | | |
| rebaudioside) | | | | |
| Water | QS | QS | QS | QS |
| | to 100 | to 100 | to 100 | to 100 |

### Example 2

Example Compositions A-C and Comparative Composition 1 from Example 1 were evaluated for chemical and physical stability per ICH accelerated aging/stress guidelines. Table 2 shows that Example Compositions A-C and Comparative Composition 1 exhibited acceptable stability in terms soluble fluoride (ppm) and soluble tin (%) and were suitably buffered to maintain a pH of 6.5 to 7.5 under accelerated shelf-life conditions. Regarding soluble tin, Example Compositions A-C, which contained sodium cocoyl glutamate and were free of sodium lauryl sulfate (SLS) appear to provide directionally higher soluble tin over the high temperature/stress conditions as compared to Comparative Composition 1, which contained sodium lauryl sulfate.

TABLE 2

| | | Comp. | Ex. | Ex. | Ex. |
|---|---|---|---|---|---|
| | | 1 | A | B | C |
| pH (10% | Initial | 7.2 | 7.1 | 7.1 | 7.0 |
| Soln.) | 4 wks-40 C./75% RH | 7.3 | 7.1 | 7.1 | 7.1 |
| | 8 wks-40 C./75% RH | 7.2 | 7.1 | 7.1 | 7.2 |
| | 13 wks-30 C./65% RH | 7.2 | 7.2 | 7.2 | 7.1 |
| | 13 wks-40 C./75% RH | 7.3 | 7.2 | 7.3 | 7.2 |
| Sol. | Initial | 1120 | 1088 | 1133 | 1083 |
| Fluoride | 4 wks-40 C./75% RH | 1030 | 1022 | 1110 | 1019 |
| (ppm) | 8 wks-40 C./75% RH | 1011 | 1010 | 1080 | 1001 |
| | 13 wks-30 C./65% RH | 1050 | 991 | 1033 | 998 |
| | 13 wks-40 C./75% RH | 990 | 979 | 1003 | 987 |
| Total | Initial | 0.36 | 0.35 | 0.36 | 0.35 |
| Tin (%) | | | | | |
| Sol. | Initial | 0.27 | 0.27 | 0.27 | 0.28 |
| Tin (%) | 4 wks-40 C./75% RH | 0.22 | 0.25 | 0.24 | 0.25 |
| | 8 wks-40 C./75% RH | 0.21 | 0.24 | 0.22 | 0.23 |
| | 13 wks-30 C./65% RH | 0.23 | 0.24 | 0.23 | 0.23 |
| | 13 wks-40 C./75% RH | 0.21 | 0.24 | 0.23 | 0.23 |

### Example 3

Example Compositions A-C and Comparative Composition 1 were also evaluated for consumer appeal during brushing. Specifically, the foaming characteristics of Example Compositions A-C and Comparative Composition 1 were quantified by means of a lab instrument called a SITA foam tester. The SITA foam tester provides automated agitation of diluted toothpaste preparations to mimic the mechanical action experienced during brushing, which correlates with sensory ratings by expert (trained) panelists. The SITA foam tester quantifies foam generation and decay rate of the foam by utilizing an array of needles that monitor changes in electro-conductance. The change in foam height is reported by the instrument in ten second intervals up to 120 seconds after a pre-determined amount of agitation has been applied to the test solution. Similarly, foam decay is measured in ten second intervals up to one minute but with no further agitation of the solution. The results shown in Table 3 are reported for the average foam height after 3 separate runs measured during foam generation at 30, 60, and 120 seconds of agitation and foam height after one minute of decay with agitation removed.

As seen in Table 3, Example Compositions A-C exhibited parity in foam volume to Comparative Composition 1, which is desirable as sodium lauryl sulfate (SLS) generally facilitates excellent foam volume for dentifrices during brushing. Unexpectedly and beneficially, Example Compositions A-C exhibited a directional improvement in foam stability over Comparative Composition 1 as indicated by the “decay” phase. Foam volume remained generally constant for Example Compositions A-C and Comparative Composition 1 between 120 seconds time and “decay,” where there is no additional agitation of the toothpaste solution for 60 seconds.

TABLE 3

| Composition | Comp. 1 | Ex. A | Ex. B | Ex. C |
|---|---|---|---|---|
| Foam Height (ml) - 30 sec | 258 | 237 | 249 | 254 |
| Foam Height (ml) - 60 sec | 342 | 319 | 323 | 336 |
| Foam Height (ml) - 120 sec | 342 | 347 | 355 | 368 |
| Foam Height (ml) - Decay | 362 | 347 | 355 | 368 |
| (180 sec with no agitation | | | | |
| after 120 sec) | | | | |

### Example 4

Example Compositions A-C and Comparative Composition 1 were evaluated to assess the metal uptake associated with each composition. Metal uptake was measured utilizing representative hard and soft tissue substrates with bovine enamel and Vitroskin assays, respectively. These established in vitro methods demonstrate that Example Compositions A-C had either directionally or statistically greater tin uptake compared to Comparative Composition 1, as shown in Tables 4 and 5.

TABLE 4
Tin Uptake on Hard Tissue

| | | Statistical |
|---|---|---|
| Sample | Avg μg/cm2 | Comparison* |
| Comparative Composition 1 | 1.20 +/− 0.10 | A |
| Example Composition A | 1.33 +/− 0.12 | AB |
| Example Composition B | 1.48 +/− 0.07 | B |
| Example Composition C | 1.62 +/− 0.05 | C |

*Means that do not share common letter = Sign. Diff @95% CI; Tukey method, N = 3 per cell

TABLE 5
Tin Uptake on Soft Tissue

| | | Statistical |
|---|---|---|
| Sample | Avg μg/cm² | Comparison* |
| Comparative Composition 1 | 2.10 +/− 0.12 | A |
| Example Composition A | 2.36 +/− 0.10 | B |
| Example Composition B | 2.44 +/− 0.10 | B |
| Example Composition C | 2.39 +/− 0.14 | B |

*Means that do not share common letter = Sign. Diff @95% CI; Tukey method, N = 3 per cell

### Example 5—Antibacterial Effect

Comparative Composition 1 and Example Composition B were evaluated by in vitro methods to determine antibacterial performance. Two key in vitro tests are University of Manchester model and Plaque Glycolysis model, which are described below.

University of Manchester Model: The anaerobic model (UoM) is used to provide a more sensitive indication of potential efficacy of the formula. In this model, saliva was collected from four healthy volunteers and pooled together for use as inoculum. Each sample is treated in triplicate twice a day for 8 days. Biofilm is recovered after 16 treatments to measure for ATP (RLU) as an end point for viable bacteria. Oral care compositions demonstrating lower APT scores provide more effective antibacterial performance. Finally, for the UoM studies, a commercial dentifrice containing NaF and KNO₃ was used as the negative control.

As seen in Table 5, Example Composition B demonstrated a statistically significant improvement in controlling anaerobic biofilm as compared to Comparative Composition 1. Both Comparative Composition 1 and Example Composition B demonstrate statistically significant improvement relative to the negative control.

TABLE 5
Viable Bacteria as ATP (RLU) - Manchester Model

| | | Statistical |
|---|---|---|
| Sample | Avg Log RLU | Comparison* |
| Commercial toothpaste with sodium | 4.93 +/− 0.20 | A |
| fluoride (negative control)** | | |
| Comparative Composition 1 | 4.29 +/− 0.16 | B |
| (Stannous Fluoride toothpaste with | | |
| sodium lauryl sulfate) | | |
| Example Composition B | 3.90 +/− 0.20 | C |
| (Stannous Fluoride toothpaste with | | |
| sodium cocoyl glutamate) | | |

*Means that do not share common letter = Sign. Diff @95% CI, Tukey method, N = 26 per cell
**Negative Control, NaF, KNO3 formula

Plaque glycolysis Model: Indirectly measures biofilm health using an in vitro adaptation of the Plaque Glycolysis Model discussed in Donald J. White, et. al., Journal of Clinical Dentistry, #6 Special Issue, pp. 59-70, (1995), the contents of which are herein incorporated by reference in its entirety for all purposes. Briefly, the method quantifies the glycolytic effects of the dentifrice on treated in vitro biofilm pool of both anaerobic and aerobic bacteria. The efficacy of each oral care composition is determined based on biofilm pH change. A lower average pH change indicates a reduction of viable bacteria and greater antibacterial performance of the respective test dentifrice. Finally, a commercial dentifrice containing NaF and KNO₃ actives was used as the negative control.

As seen in Table 6, Example Composition B was statistically equivalent to Comparative Composition 1 at reducing a general oral bacteria population. Both Example Composition B and Comparative Composition 1 performed significantly better than the negative control at controlling the bacterial biofilm.

TABLE 6
Plaque Glycolysis Study

| | | Statistical |
|---|---|---|
| Sample | Avg pH Change | Comparison* |
| Commercial toothpaste with sodium | 2.14 +/− 0.02 | A |
| fluoride (negative control)** | | |
| Comparative Control 1 (Stannous | 1.58 +/− 0.02 | B |
| Fluoride toothpaste with sodium lauryl | | |
| sulfate) | | |
| Example Composition B (Stannous | 1.53 +/− 0.04 | B |
| Fluoride toothpaste with sodium | | |
| cocoyl glutamate) | | |

*Means that do not share common letter = Sign. Diff @95% CI, Tukey method, N = 3 per cell
**Negative Control, NaF, KNO₃ formula

### Example 6

Two comparative compositions (Comparative Compositions 2 and 3) were prepared in the form of dentifrices. The formulation for Comparative Compositions 2 and 3 are shown in Table 8. The formulation of Example Composition B and Comparative Composition 1 from Example 1 are also reproduced in Table 8:

TABLE 8
Toothpaste Formulations Tested in pH Cycling Study

| | | Comp. | Comp. | Comp. |
|---|---|---|---|---|
| | Ex. B | 1 | 2 | 3 |
| US INCI Compound Name | (Wt. %) | (Wt. %) | (Wt. %) | (Wt. %) |
| Hydrated Silica | 21.5 | 21.5 | 21.5 | 21.5 |
| Water | 21 | 19 | 19 | 19 |
| Glycerin | 6 | 6 | 6 | 6 |
| PEG-12 | 2 | 2 | 2 | 2 |
| Tetrasodium Pyrophosphate | 2 | 2 | 2 | 2 |
| Sodium Lauryl Sulfate Powder | | 1.5 | 1.5 | 1.5 |
| Sodium Cocoyl Glutamate | 0.46 | | | |
| Cocamidopropyl Betaine | 0.9 | 0.38 | 0.38 | 0.38 |
| Alkyl Polyglucoside | 0.75 | | | |
| Sodium Carboxymethyl | 1.8 | 1.8 | 1.8 | 1.8 |
| Cellulose (microcrystalline | | | | |
| and/or non-microcrystalline) | | | | |
| Zinc Phosphate Monohydrate | 1 | 1 | 1 | 1 |
| Titanium Dioxide | 0.5 | 0.5 | 0.5 | 0.5 |
| Stannous Fluoride or Sodium | 0.45 | 0.45 | | 0.24 |
| Fluoride | | | | |
| Xanthan Gum | 0.3 | 0.3 | 0.3 | 0.3 |
| Flavor (Flavoring Agents, | 1.7 | 1.7 | 1.7 | 1.7 |
| Sucralose, and Sodium Saccharin) | | | | |
| pH adjuster (Citric Acid and | 1.2 | 1.2 | 1.2 | 1.2 |
| Trisodium Citrate Dihydrate | | | | |
| Sorbitol (70 Wt. % Soln. Of | QS | QS | QS | QS |
| Sorbitol) | to 100 | to 100 | to 100 | to 100 |
| Formula pH (10% Soln. In Water) | 7.3 | 7.3 | 7.4 | 7.5 |

Example Composition B and Comparative Compositions 1-3 were evaluated for their ability to prevent enamel loss and demineralization upon repeated acid challenge on an enamel substrate. Microhardness was used as a before and after marker for erosion protection. Specifically, a 5-day cycling study using an automated robotic system was performed to determine the relative erosion protection potential of dentifrices containing stannous fluoride or sodium fluoride as a fluoride source, and either sodium lauryl sulfate or sodium methyl cocoyl taurate as surfactant, compared to a control dentifrice containing sodium lauryl sulfate surfactant and lacking fluoride.

Disk Creation

Specifically, to assess erosion protection, each dentifrice was applied to two disks, with each disk comprising 10 bovine cores affixed to each disk. One hundred 3 mm bovine rods were cut down to the appropriate size to form the cores. The rods (cores) came with an excess of clear acrylic that was cut off close to the point where white acrylic meets clear acrylic using a blade. The rods were then placed on the disks, sanded down, and polished. The enamel sides of the rods were sanded down and polished in stages, making sure that at all stages the disks are flush and even. While grinding, water was flowing over the surfaces, with a small amount of soap being used as necessary for slightly reducing the friction of the circulating pad. The cores were polished with 320, 400, 800 and then 1200 grit, using the following parameters: Time=2 minutes; Platen Speed=180 rpm; Head Speed=60 rpm; Fluids=Fresh Water; Head Rotation=Contra Rotation; Mode=Single; and Force=1 psi.

Once a desired surface was reached, the cores were polished with 6 μm Yellow MetaDi Diamond suspension polishing fluid on a white Trident polishing pad using the following parameters: Time=6 minutes; Platen Speed=180 rpm; Head Speed=60 rpm; Fluids=Off; Head Rotation=Contra Rotation; Mode=Single; and Force=2 psi. At 2 minutes elapsed time, additional 6 μm Yellow MetaDi Diamond suspension polishing fluid was added onto the rotating platen. At 4 minutes elapsed time, 6 grams of 0.05 μm white alumina polishing fluid was added onto the rotating platen.

Microhardness Readings

Microhardness readings were taken to quantify the efficacy of the tested dentifrice compositions against enamel erosion. The readings were taken before a 5% citric acid, post etch, and then after the pH cycling (Demin/Remin). A MicroMet 6020 microindentation hardness tester running Omnimet software was used to take hardness readings. The instrument makes an indent in the shape of a diamond (0). The width from the left most point to the right most point is measured. This measurement is known as the hardness amount and is given in HK values. For each bovine core, 3 measurements were made and were in the middle of the core.

5% Citric Acid Etch

The disks were etched in a 5 wt. % citric acid solution before the pH cycling began. A 200 ml solution of 5 wt. % citric acid was made and then poured into a beaker. Each disk was individually placed in a 5 wt. % citric acid solution for 30 seconds, making sure each disk was fully submersed. After the 30 seconds, the disks were washed in deionized water and then patted dry with a delicate wipe. Once all the disks were treated, they were measured again on the MicroMet6020, and robotic cycling was commenced.

5 Day Robotic pH Cycling

Steps 1-27, shown in Table 7, were programmed and run robotically in consecutive order.

TABLE 7

| | | | |
|---|---|---|---|
| 1 | Rinse | 6 | Seconds |
| 2 | Saliva | 1 | hour |
| 3 | Rinse | 6 | seconds |
| 4 | Treatment (Toothpaste) | 2 | minutes |
| 5 | Rinse | 3 | seconds |
| 6 | Saliva | 1 | hour |
| 7 | Rinse | 6 | seconds |
| 8 | Acid Challenge | 2 | minutes |
| 9 | Rinse | 3 | seconds |
| 10 | Saliva | 1 | hour |
| 11 | Rinse | 6 | seconds |
| 12 | Acid Challenge | 2 | minutes |
| 13 | Rinse | 3 | seconds |
| 14 | Saliva | 1 | hour |
| 15 | Rinse | 6 | seconds |
| 16 | Acid Challenge | 2 | minutes |
| 17 | Rinse | 3 | seconds |
| 18 | Saliva | 1 | hour |
| 19 | Rinse | 6 | seconds |
| 20 | Acid Challenge | 2 | minutes |
| 21 | Rinse | 6 | seconds |
| 22 | Saliva | 1 | hour |
| 23 | Rinse | 6 | seconds |
| 24 | Treatment | 2 | minutes |
| 25 | Rinse | 3 | seconds |
| 26 | Saliva | 16-18 | hours |
| 27 | Rinse | 6 | seconds |

Example Composition B and Comparative Compositions 1-3 were evaluated using a pH cycling model. Specifically, the pH cycling test model included 20 runs (n=20) for each oral care composition. The two stannous fluoride toothpastes described in Table 8 (Comparative Composition 1 and Example Composition B) were compared to a control toothpaste without fluoride (Comparative Composition 2) and also to a toothpaste containing the same 1100 ppm amount of fluoride (Comparative Composition 3). Microhardness measurements using 50 g force for 5 seconds (HK) were made with each test sample. The percentage of demineralization was then calculated based on Knoop microhardness values (HK) of pre-treated and post-cycling enamels in each group using the following equation:

% Demineralization=100*(HK_{pre-treated}−HK_{post-cycling})/HK_{pre-treated}.

Toothpastes providing a lower value more effectively protect the enamel. As seen in Table 9, Comparative Composition 2 was less effective in preventing acid erosion while Comparative Compositions 1 and 3 and Example Composition B were overall more effective in maintaining enamel hardness and preventing demineralization.

TABLE 9

| | Microhardness value ± SEM | | | |
|---|---|---|---|---|
| | | Pre- | Post- | % Demineral- |
| Groups | Baseline | treated | cycling | ization |
| Comparative | 303.1 ± 4.7 | 226.1 ± 6.5 | 68.4 ± 2.1 | 69.7 ± 0.6 ^{A} |
| Composition 3 | | | | |
| Comparative | 302.3 ± 5.9 | 227.5 ± 6.7 | 78.5 ± 2.3 | 65.5 ± 0.7 ^{B} |
| Composition 4 | | | | |
| Example | 311.9 ± 3.2 | 226.9 ± 6.1 | 84.2 ± 2.3 | 62.9 ± 0.4 ^{C} |
| Composition D | | | | |
| Example | 308.2 ± 5.0 | 226.7 ± 3.6 | 92.2 ± 2.7 | 59.3 ± 1.0 ^{D} |
| Composition E | | | | |

% Demineralization sharing the same superscript letters are not significantly different

1. An oral care composition comprising:
a surfactant system comprising:
an amino acid derived surfactant, and
an amphoteric surfactant;
a zinc salt; and
a stannous ion source.

2. The oral care composition according to claim 1, wherein the amino acid derived surfactant is a glutamic acid derived surfactant.

3. (canceled)

4. The oral care composition according to claim 2, wherein the aliphatic carbon chain of the acyl glutamic acid derived surfactant has 4 to 19 carbons.

5. The oral care composition according to claim 1, wherein the amino acid derived surfactant comprises sodium cocoyl glutamate.

6. The oral care composition according to claim 1, wherein the surfactant system is present in an amount from about 0.5 to about 9 wt. %, based on the total weight of the oral care composition.

7. The oral care composition according to claim 1, wherein the amino acid derived surfactant is present in an amount from about 0.1 to about 4 wt. %, based on the total weight of the oral care composition.

8. The oral care composition according to claim 1, wherein the amphoteric surfactant is present in an amount from about 0.3 to about 8 wt. %, based on the total weight of the oral care composition.

9. The oral care composition according to claim 1, wherein the oral care composition is formulated to have a weight ratio of the total amount of amino acid derived surfactant to the total amount of amphoteric surfactant from about 1:1 to about 1:8.

10. The oral care composition according to claim 1, wherein the zinc salt is selected from zinc phosphate, zinc citrate, zinc oxide, zinc lactate, zinc pyrophosphate, zinc sulfate, zinc chloride, and a combination of two or more thereof.

11. The oral care composition according to claim 1, wherein the zinc salt comprises zinc phosphate.

12. The oral care composition according to claim 1, wherein the stannous ion source comprises stannous pyrophosphate, stannous fluoride, stannous chloride, stannous acetate, and a combination of two or more thereof.

13. The oral care composition according to claim 1, wherein the stannous ion source comprises stannous fluoride.

14. The oral care composition according to claim 1 further comprising:
a fluoride ion source.

15. The oral care composition according to claim 14, wherein the fluoride ion source is selected from stannous fluoride, sodium fluoride, potassium fluoride, sodium monofluorophosphate, sodium fluorosilicate, ammonium fluorosilicate, amine fluoride, ammonium fluoride, and a combination of two or more thereof.

16. The oral care composition according to claim 1 further comprising:
a silica abrasive, optionally, in an amount ranging from about 1 to about 55 wt. %, based on the total weight of the oral care composition.

17. The oral care composition according to claim 16, wherein the silica abrasive has a N₂ BET surface area of less than 50 m²/g and an Einlehner hardness of from 4 to 11, and an oil absorption of from 80 cc/100 g to 100 cc/100 g.

18. The oral care composition according to claim 1 further comprising:
a thickening agent, optionally, present in an amount from about 0.5 to about 20 wt. %, based on the total weight of the oral care composition.

19. The oral care composition according to claim 18, wherein the thickening agent is selected from a polysaccharide, a silica thickener, an acrylate, and a combination of two or more thereof.

20. The oral care composition according to claim 18, wherein the thickening agent comprises a silica thickener and a thickening agent selected from cellulose, xanthan gum, diutan gum, carrageenan, gellan gum, welan gum, pectin, sclerotium gum, starch, galactoarabinan, and a combination of two or more thereof.

21. An oral care composition comprising:
from about 0.1 to about 4 wt. % of a surfactant system comprising:
an amino acid derived surfactant, and
an amphoteric surfactant;
a zinc salt;
a stannous ion source;
from about 5 to about 35 wt. % of a silica abrasive;
from about 10 to about 50 wt. % of a polyol; and
from about 0.5 to about 20 wt. % of a thickening agent.
wherein all weight percentages are based on the total weight of the oral care composition.
